(19) United States
(12) Patent Application Publication
(10) Pub. No.: US 2002/0099506 A1
(43) Pub. Date: Jul. 25, 2002

(54) Methods and apparatus for predicting ligand binding interactions

(76) Inventors: Wely B. Floriano, Pasadena, CA (US); Nagarajan Vaidehi, Arcadia, CA (US); William A. Goddard III, Pasadena, CA (US)

Correspondence Address: SCOTT HARRIS, Fish & Richardson P.C., Suite 500, 4350 La Jolla Drive, San Diego, CA, 92122, US

(21) Appl. No.: 10/010,725

(22) Filed: Nov. 30, 2001

Related U.S. Application Data
(63) Continuation of application No. 09/816,772, filed on Mar. 23, 2001, now abandoned.
(60) Provisional application No. 60/191,895, filed on Mar. 23, 2000.
(60) Provisional application No. 60/213,658, filed on Jun. 23, 2000.

(51) Int. Cl. G06G 7/48; G06G 7/58; G06F 19/00; G01N 33/48; G01N 33/50

(52) U.S. Cl. 702/19; 703/11

(57) ABSTRACT

Computer-implemented methods and apparatus implement a hierarchy of molecular modeling techniques for predicting binding sites of ligands in proteins, designing new pharmaceuticals and understanding the interactions of proteins involved in microbial pathogens. The techniques employ a hierarchical strategy ranging from coarse grain to fine grain conformational search methods combined with hierarchical levels of accuracy in scoring functions.

### CROSS-REFERENCE TO RELATED APPLICATIONS

This application is a continuation of and claims priority to U.S. application Ser. No. 09/816,772, filed Mar. 23, 2001 which claims the benefit of U.S. Provisional Application No. 60/191,895, filed Mar. 23, 2000 and No. 60/213,658, filed Jun. 23, 2000. Each of these prior applications is incorporated by reference herein.

### STATEMENT REGARDING FEDERALLY SPONSORED RESEARCH AND DEVELOPMENT

The U.S. Government has certain rights in this invention pursuant to Grant No. DAAG55-98-1-0266 awarded by the Department of the Army.

### FIELD OF THE INVENTION

The present invention relates to computational methods for predicting ligand binding sites in proteins, modeling protein-ligand interactions and drug design, and to computer-implemented apparatus for performing such computations.

### BACKGROUND

Computational techniques for evaluating protein-ligand interactions are known and can provide useful information about the functions of proteins. B. K. Shoichet et al. (1993) Science 259, 1445-1450; P. Burkhard et al. (1999) J. Mol. Biol. 277, 449-466. However, although these techniques may provide explanations for the interactions between known ligands and receptors, they generally fail in reliably predicting the binding site of an endogenous ligand to its receptor as well as binding affinities of untested ligands. These two factors are critical in predicting function of proteins and drug design.

Most docking procedures in the literature, such as T. A. Ewing, et al. (1997) J. Comput. Chem. 18, 1175-1189, and G. M. Morris, et al. (1998) J. Comput. Chem. 14, 1639-1662, use Monte Carlo methods or orientation matching algorithms for fast conformational search. In such procedures, scoring functions are based only on non-bond energies and typically do not include salvation. As a result, the binding energies calculated using these methods are not accurate enough for selecting the best binding configuration of a particular ligand or for comparing the binding affinities of different ligands to the same protein target. Many known scoring functions neglect salvation effects that can be critical in scoring configurations or calculating binding energies. One exception is a recently reported dock procedure that includes salvation effects using a Generalized Born model of continuum salvation, X. Q. Zou, et al., J. Am. Chem. Soc. 121, 8033-8043 (1999), but this procedure is both memory inefficient and time consuming. In other methods, the scoring function involves salvation for the ligand only. B. K. Soichet, et al. (1999) Proteins: Structure, Function and Genetics 34, 4-16. Still other techniques use free energy perturbation methods to calculate binding free energies, but these techniques are computationally demanding and are not effective as fast screening techniques. A. J. McCammon, et al., in Dynamics of Proteins and Nucleic Acids, Cambridge; New York: Cambridge University Press (1987). In summary, while they may be useful for some purposes, methods using a single scoring function with one docking method are generally inadequate to predict the binding site in a protein, especially when there is no knowledge of that binding site.

As a result, there is a need for computer modeling techniques that provide a fast and efficient means to predict the ligand binding site in a protein, to calculate the binding free energies of potential ligands, and to perform screening of large virtual libraries.

### SUMMARY OF THE INVENTION

The invention provides a hierarchy of molecular modeling techniques and apparatus for predicting binding sites of ligands in proteins, designing new pharmaceuticals and understanding the interactions of proteins involved in microbial pathogens. These techniques generally employ a hierarchical strategy ranging from coarse grain to fine grain conformational search methods combined with hierarchical levels of accuracy in scoring functions. Various implementations of the invention use hierarchical combinations of coarse-grain docking methods, fine grain molecular dynamics techniques and scoring functions with different levels of accuracy that include salvation effects, to provide computationally-efficient and accurate models for predicting binding site of ligands in proteins and drug design. These protocols emphasize a hierarchical strategy of scoring a large set of coarse grain docked structures with an all atom forcefield scoring function with salvation. Subsequently, a subset of these structures is used for fine grain annealing molecular dynamics (MD) methods, which include salvation effects and an all atom forcefield, such as AMBER, CHARMM, DREIDING or MM3. The combination of these techniques provides for consistent and reliable predictions of the binding site for ligands in proteins.

The methods and apparatus described herein are useful in a wide variety of applications, such as performing fast screening of virtual chemical compound libraries against targets of pharmacological interest, fast scanning of both globular and membrane bound proteins for potential binding sites, prediction of potential ligands and ligand binding modes, and prediction of receptor function based on selective binding affinities. These methods can also be used in identifying the interaction of cellular receptors with surface structures expressed by microbial pathogens to understand the molecular basis of pathogenesis.

In general, in one aspect, the invention features methods and apparatus, including computer program apparatus, implementing techniques for modeling ligand-protein binding interactions. The techniques can include providing structural information describing the structure of a protein and a set of one or more ligands; using the structural information for the protein to identify a binding region of the protein; identifying a plurality of preferred binding conformations for each of the set of ligands in the binding region; optimizing the preferred binding conformations using annealing molecular dynamics including salvation effects; calculating a binding energy for each of the set of ligands in the corresponding optimized preferred binding conformations; and selecting for each of the set of ligands the lowest calculated binding energy in the optimized preferred binding conformations, and outputting the selected calculated binding energies as the predicted binding energies for each of the set of ligands.

Particular implementations of the invention can include one or more of the following features. The binding region can be a known binding region defined by the structural information or an unknown binding region. If the binding region is unknown, using the structural information for the protein to identify a binding region of the ligand in the protein can include predicting a probable binding region based at least in part on the structural information. Predicting a probable binding region can include mapping the empty volumes available for ligand binding in the protein to identify one or more potential binding regions; generating initial conformations for one or more ligands known to bind the protein using docking techniques in each of the one or more potential binding regions; selecting from the initial conformations for each of the known ligands a plurality of best conformations in each of the potential binding regions and scoring an energy function for each of the best conformations; and identifying the probable binding site based on a spatial location of the conformations having the lowest energy scores. The techniques can further include before scoring the energy function for each of the best conformations, optimizing the selected best conformations to obtain a set of energy-minimized conformations for each of the known ligands in each of the potential binding regions where the energy function can be scored for each of the energy-minimized conformations. The techniques can further include before scoring the energy function for each of the best conformations, calculating for each of the best conformations a percentage of the ligand surface area buried within the protein for the conformation, where the energy function can be scored only for a subset of the best conformations having a calculated percentage of the ligand surface area buried within the protein exceeding a predetermined surface area threshold. The preferred binding conformations for each of the set of ligands can be identified by generating initial conformations for each of the set of ligands in the binding region using docking techniques, and selecting from the initial conformations for each of the ligands a plurality of best conformations. The techniques can further include after selecting the best conformations, optimizing the selected best conformations to obtain a set of energy-minimized conformations for each of the ligands, where the preferred binding conformations can include the energy-minimized conformations. The annealing molecular dynamics can include a full atom force field. The salvation effects can include a continuum description of salvation or a surface-area based solvation model. Calculating a binding energy for each of the set of ligands can include taking the difference in the ligand energy in the receptor and in solution. The binding energy can be calculated for a ligand according to a scoring function comprising subtracting the free energy of the ligand in water from the energy of the ligand in the protein. The binding energy can be calculated for a ligand according to a scoring function comprising subtracting the free energy of the protein and the free energy of the ligand from the free energy of the ligand in the protein. The techniques can further include identifying from the set of ligands one or more ligands predicted to have high binding affinity based on the calculated binding energy of the ligands in the binding site. The protein can be a globular protein or a transmembrane protein.

In general, in another aspect, the invention features methods and apparatus, including computer program apparatus, implementing techniques for predicting the structure of a protein binding site for a protein having an unknown binding site. The techniques can include providing structural information describing the structure of a protein having an unknown binding site and a set of one or more ligands known to bind to the protein; using the structural information for the protein to identify a plurality of potential binding regions of the protein; generating initial conformations for one or more of the ligands using docking techniques in each of the potential binding regions; selecting from the initial conformations for each of the ligands a plurality of best conformations in each of the potential binding regions and scoring an energy function for each of the best conformations; identifying the probable binding site based on a spatial location of the conformations having the lowest energy scores; and outputting structure information describing the three-dimensional structure of the probable binding site.

Particular implementations can include one or more of the following features. The techniques can further include before scoring the energy function for each of the best conformations, optimizing the selected best conformations to obtain a set of energy-minimized conformations for each of the ligands in each of the potential binding regions, where the energy function is scored for each of the energy-minimized conformations. The techniques can further include before scoring the energy function for each of the best conformations, calculating for each of the best conformations a percentage of the ligand surface area buried within the protein for the conformation, where the energy function is scored only for a subset of the best conformations having a calculated percentage of the ligand surface area buried within the protein exceeding a predetermined surface area threshold.

In general, in another aspect, the invention features methods and apparatus, including computer program apparatus, implementing techniques for screening a ligand library. The techniques can include receiving protein structural information describing the structure of a protein; receiving ligand structural information describing the structure of a plurality of ligands in a ligand library; receiving an input specifying a desired number of candidate ligands to be identified in the ligand library; using the structural information for the protein to identify a binding region of the protein; generating a set of initial binding conformations for each of the ligands in the binding region; calculating an energy function for each of the initial binding conformations and selecting for each of the ligands a plurality of the initial binding conformations having the lowest calculated energy as a set of best conformations; optimizing the best conformations; calculating a binding energy for each of the ligands in the corresponding optimized best conformations; and selecting from the plurality of ligands a set of the desired number of candidate ligands having the lowest calculated binding energy in the optimized best binding conformations, and outputting the selected set of candidate ligands.

Particular implementations can include one or more of the following features. The plurality of ligands can include at least 500, 1,000, 5,000, 10,000, 50,000, or 100,000 or more ligands. Calculating a binding energy for each of the set of ligands can include taking the difference in the ligand energy in the receptor and in solution. The binding energy can be calculated for a ligand according to a scoring function comprising subtracting the free energy of the ligand in water from the energy of the ligand in the protein.

In general, in another aspect, the invention features computational models of a ligand-protein complex for a protein having an unknown binding site. The models can include a computer-readable memory storing data describing an optimized preferred binding conformation for the protein and a ligand known to bind to the protein. The optimized binding conformation can be generated according to the methods described above.

In general, in another aspect, the invention features methods and apparatus, including computer program apparatus, implementing techniques for generating and using pharmacophores. The techniques can include providing structural information describing the structure of a protein and a set of one or more ligands known to bind to the protein; using the structural information for the protein to identify a binding region of the protein; identifying a plurality of preferred binding conformations for each of the set of ligands in the binding region; optimizing the preferred binding conformations using annealing molecular dynamics, the annealing molecular dynamics including salvation effects; calculating a binding energy for each of the set of ligands in the corresponding optimized preferred binding conformations; selecting for each of the set of ligands the optimized preferred binding conformation having the lowest calculated binding energy; generating a pharmacophore model based at least in part on the selected optimized preferred binding conformations, the pharmacophore model defining a pattern of ligand features predicted to be required for binding to the protein; and outputting data representing the pharmacophore model for use in drug design. Pharmacophores generated according to these techniques can be used, for example, as a templates in the identification and/or design of lead compounds predicted to bind to the protein.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a flow diagram illustrating a general computational protocol for modeling ligand-protein interactions according to the invention.

FIG. 2 is flow diagram illustrating a particular implementation of the protocol of FIG. 1 in more detail.

FIG. 3 is a block diagram illustrating a computer system for running a docking protocol according to the invention.

FIG. 4 is a flow diagram illustrating an implementation of a docking protocol for fast and high throughput virtual screening of large databases of chemical compounds.

FIG. 5 is a graphical representation of a comparison of the known binding site for phenylalanine in phenylalanyl t-RNA synthetase and a predicted binding site generated according to the method of FIG. 2.

FIG. 6 illustrates a set of phenylalanine analogs for which binding energies in the phenylalanyl t-RNA synthetase binding site of FIG. 5 were calculated according to the method of FIG. 2.

FIG. 7 is a graph of binding energies in phenylalanyl t-RNA synthetase calculated for the phenylalanine analogs of FIG. 6.

FIG. 8 is a graphical representation of the predicted binding site for histidine in histidyl t-RNA synthetase generated according to the method of FIG. 2.

FIG. 9 is a graph of binding energies in histidyl t-RNA synthetase for a set of amino acid ligands generated according to the method of FIG. 2.

FIG. 10 is a graph of binding energies in olfactory receptor S25 for a series of alcohol and acid ligands generated according to the method of FIG. 2.

FIG. 11 is a graphical representation of the predicted binding site for hexanol and heptanol in olfactory receptor S25 generated according to the method of FIG. 2.

FIG. 12 is a graph of binding energies in olfactory receptor S18 for a series of alcohol and acid ligands using the method of FIG. 2.

FIG. 13 is a graphical representation of the predicted binding site for nonanol in olfactory receptor S18 generated according to the method of FIG. 2.

FIG. 14 is a graphical representation illustrating the superposition of the experimentally determined and predicted structures of cis-retinal bound to bovine rhodopsin.

### DETAILED DESCRIPTION

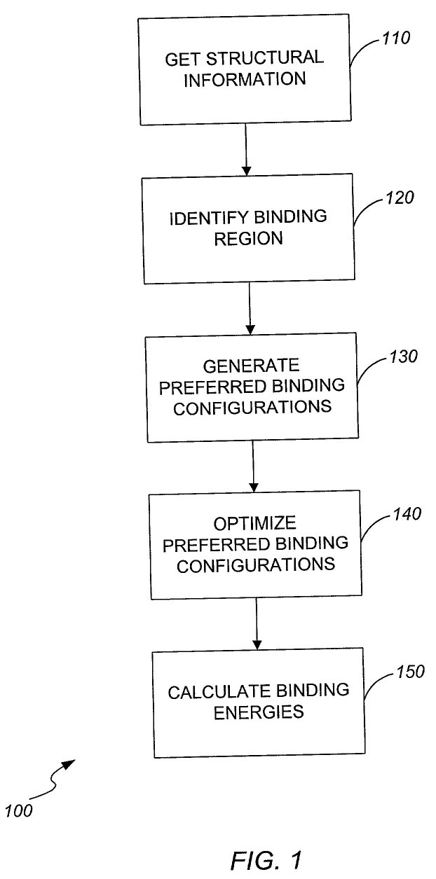

The present invention provides computational modeling techniques for modeling protein-ligand binding interactions. In one embodiment, illustrated in FIG. 1, a modeling protocol 100 starts by obtaining structural information describing a protein and a set of one or more potential ligands (step 110). If the binding site of the ligand in the protein is not known, the method maps the potential ligand binding sites and identifies a probable binding site (step 120). The method applies coarse-grained docking algorithms (e.g., known Monte Carlo or matching techniques) to generate a set of configurations for each ligand in the known or predicted binding site (step 130). The method then applies molecular mechanics and annealing molecular dynamics techniques (including salvation effects) to a selected subset of the resulting ligand-protein complexes (step 140) and calculates binding affinities for each of the potential ligands (step 150).

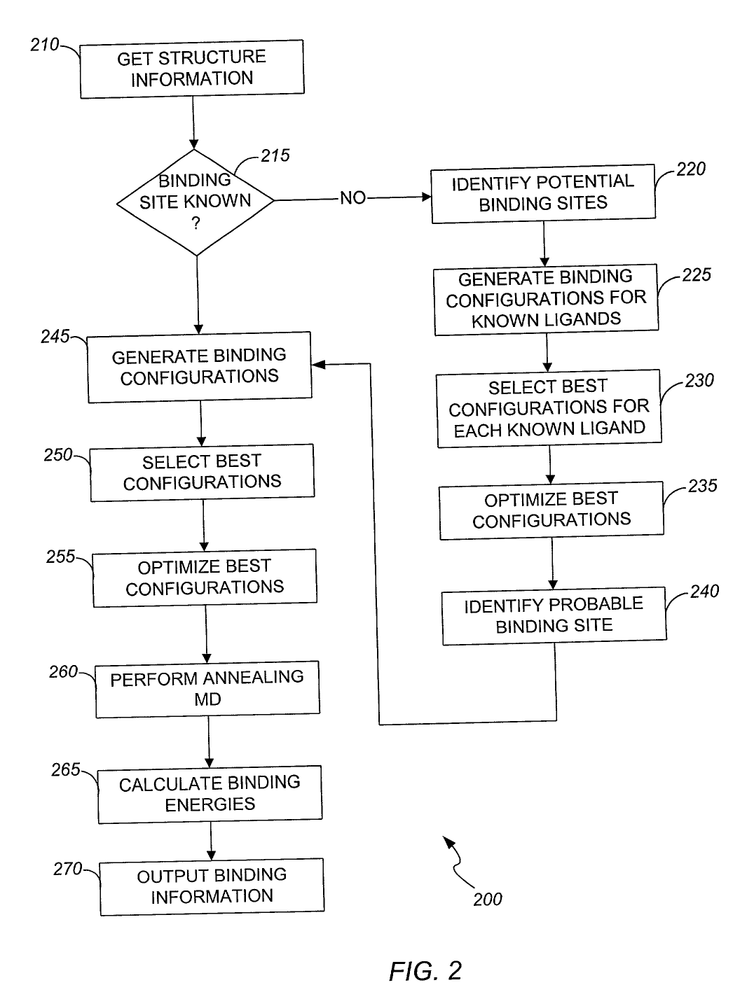

FIG. 2 provides a more detailed illustration of one implementation of the protocol described above. The method 200 begins by retrieving structural information for a protein and a set of potential ligands (step 210). Such information can be retrieved as a set of three-dimensional coordinates defining atomic positions for each atom or group of atoms in the protein or ligand, in the form of, for example, a data file in a standard file format such as the Protein Data Bank (PDB) format for protein structural information and/or the Crystallographic Information File (CIF) format used by the Cambridge Structural Database for organic and metal organic ligands. Ligand structural information can also be retrieved as, e.g., twodimensional drawings showing molecular connectivity (e.g., in the well-known Structure Data (.SD) file format), which can be converted to a three-dimensional format using standard programs, such as Sybyl Concord, available from Tripos Software, Inc. The structural information can be derived from experimentally-determined structural data (based, e.g., on data measured by techniques such as x-ray crystallography), or from computational models such as those described for G-protein coupled receptors in N. Vaidehi, et al., “Methods and Apparatus for Predicting Structure of G-Protein Coupled Receptors,” U.S. application Ser. No. 09/816,755, filed on Mar. 23, 2001, which is incorporated by reference herein, or other known techniques.

If the structure and location of the protein's binding site are not known (the NO branch of step 215), the method predicts that information as follows. Using the protein structural information, the method identifies a set of potential ligand binding sites by mapping the empty volumes available for ligand binding in the protein (step 220). The total volume available for docking is divided into small binding regions. Initial conformations for one or more ligands known to bind the protein are generated for each of the potential binding areas (step 225) using known techniques, such as the well-known DOCK 4.0 package, T. A. Ewing, et al. (1997) J. Comput. Chem. 18, 1175-1189, which is incorporated by reference herein (DOCK 4.0 is available at http://www.cmpharm.ucsf.edu/kuntz/), or any other publicly-available docking software. A set of best conformations (e.g., from about 1% to about 20% or more of the initial conformations identified in step 225, depending on the particular application) is selected for each of the known ligands in each potential binding area (step 230). These conformations are then optimized using molecular mechanics (step 235). The best of these conformations (i.e., those having the lowest energy scores) are identified and a probable binding site is identified based on the spatial clustering of the best conformations (step 240). Optionally, an additional selection criteria based on the percentage of the ligand surface area buried within the protein can be applied prior to the selection of lowest energy conformations. This probable binding site is used in the following steps.

If the binding site is known (the YES branch of step 215), or once a probable binding site has been identified, the method generates initial conformations for each of the set of ligands in the known or predicted binding site (step 245) as described for step 225 above. A subset of good conformations (e.g., from about 1% to about 20% or more of the initial conformations as described above) is selected for each of the ligands in the binding site (step 250), and these structures are then optimized using molecular mechanics (step 255). Subsequently, annealing molecular dynamics, including salvation effects as will be described below, is performed for all complexes (step 260). The best (lowest energy) conformation for each ligand is selected and binding energies are calculated for each ligand in that best conformation (step 265). The binding energies for different ligands can be compared and ordered to identify those ligands having highest affinity for the receptor. This binding energy data can be compared to the experimental affinity data measured for all the ligands or a subset thereof. Finally, the method outputs a data file containing a list of ligand-protein conformations and binding energies for each listed conformation (step 270).

The output conformations provide an atomic level model of the binding site. The residues of the protein located within 5A of the ligand can be identified for point mutation studies on the receptor. The 3D protein data bank formatted output files can be viewed using standard molecular viewer software applications, such as Quanta, Molscript or the like.

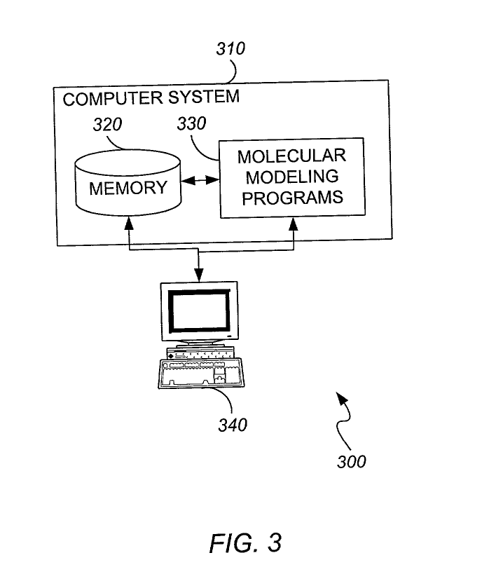

The techniques described herein can be implemented using a modeling system 300 as shown in FIG. 3. Modeling system 300 includes a general-purpose programmable digital computer system 310 of conventional construction, including a memory 320 and a processor for running a suite of one or more molecular modeling programs 330. System 300 also includes input/output devices 340, and, optionally, conventional communications hardware and software by which computer system 310 can be connected to other computer systems. Although FIG. 3 illustrates modeling system 300 as being implemented on a single computer system, the functions of system 300 can be distributed across multiple computer systems, such as on a network. Those skilled in the art will recognize that system 300 can be implemented in a variety of ways using known computer hardware and software, such as, for example, a Silicon Graphics Origin 2000 server having multiple R10000 processors running at 195 MHz, each having 4 MB secondary cache, or a dual processor Dell PowerEdge system equipped with Intel PentiumIII 866 MHz processors with 1 Gb of memory and a 133 MHz front side bus.

In a preferred embodiment, which we have called Hier-Dock, system 300 uses the structural information obtained in step 210 to calculate a negative image of the protein's molecular surface to find the available volume for ligand docking according to M. L. Connolly (1983) Science 221, 709-713, fills this volume with overlapping spheres, and divides the potential binding areas of the sphere-filled volume into overlapping regions using the DOCK 4.0 package, discussed above. Sphere clusters were generated for the whole receptor using the program SPHGEN, I. D. Kuntz, et al. (1982) J. Mol. Biol. 161, 269-288, which is incorporated by reference herein.

Starting ligand conformations were optimized by minimization of the potential energy using the conjugate gradient method with the DREIDING force field, S. L. Mayo, et al. (1990) J. Phys. Chem. 94, 8897-8909, and Gasteiger charges, J. Gasteiger, et al. (1980) Tetrahedron 36, 3219-3228, both of which are incorporated by reference herein. The minimized conformations were used as starting conformations for docking. Solvation energies for ligands were calculated using the Poisson-Boltzmann continuum solvent model with the program JAGUAR, version 4.0, available from Schrodinger, Inc., of Portland, Oregon. The DOCK 4.0 package was used to generate ligand orientations in the receptor, using flexible docking with torsion minimization of ligands, a nondistance-dependent dielectric constant of one, and a cutoff of 10 Å for energy evaluation. Conformations were ranked using energy scoring.

Annealing molecular dynamics was performed on a subset of the energy minimized ligand conformations (e.g., from about 1% to about 20% or more of these configurations) using MPSim software, K. -T. Lim, et al. (1997) J. Comput. Chem. 18, 501-521, which is incorporated by reference herein, using a full atom force field and salvation effects, such as a continuum description of the salvation using Poisson-Boltzmann method (PBF), D. J. Tannor, et al. (1994) J. Am. Chem. Soc. 116, 11875-11882, or the surface generalized Born (SGB) model, A. Ghosh, et al. (1999) J. Phys. Chem. B. 102, 10983-10990, both of which are incorporated by reference herein. Those skilled in the art will recognize that other salvation models can also be used, including, for example, empirical salvation models that estimate solvation free energies as a function of solvent accessible surface area of the protein (such as the Fast Solvation Model (FSM)), as described in R. L. Williams, et al. (1992) Proteins: Structure, Function and Genetics 14, 110-119, which is incorporated by reference herein. Annealing molecular dynamics simulations were performed in 5 to 10 cycles of 1 ps at each temperature from 50K and 600K in steps of 20K, using the DREIDING force field, a nondistance-dependent dielectric constant of one, and a nonbond list cutoff of 9 Å. Those skilled in the art will recognize that other known atomic forcefields, including, for example, the AMBER, CHARMM, or MMFF forcefields can be used in the MD simulations in place of the DREIDING forcefield described here. The best conformers from annealing were submitted to energy minimization.

In some implementations, fast scoring of large combinatorial libraries was performed using the following scoring function:

ΔΔG^{binding}=ΔG^{ligandinprotein}=ΔG^{ligandinwater}, (1)

where ΔΔG^{binding} is the free energy of binding for the ligand, ΔG^{ligandinprotein} is the free energy of the ligand in the protein and ΔG^{ligandinwater} is the free energy of the ligand in water. This scoring function calculates the binding affinity using solvation penalty for the ligand, and provides a computationally efficient route to qualitative affinity data, useful, for example, in comparisons of large numbers of ligands.

A more accurate scoring function takes into account the salvation energies of all involved entities and is given by:

ΔG^{bind}=ΔG^{protein+ligand}−ΔG^{protein}−Δ^{ligand}, (2)

where the salvation of the protein and ligand and the complex of the protein with the ligand are included explicitly. The salvation contribution to this scoring function can be calculated either using the PBF, SGB or FSM solvation models, as described in D. J. Tannor, et al. (1994) J. Am. Chem. Soc. 116, 11875-11882, A. Ghosh, et al. (1999) J. Phys. Chem. B. 102, 10983-10990, and R. L. Williams, et al. (1992) Proteins: Structure, Function and Genetics 14, 110-119, each of which is incorporated by reference herein. Free energies were calculated using MPSim with a forcefield of the user's choice and salvation. The binding energies obtained using this scoring function gives quantitative comparisons to the experimental measurements of difference in binding energies for various ligands. The binding energies for the best ligand-protein complexes were calculated as the difference in the ligand energy in the receptor and in solution.

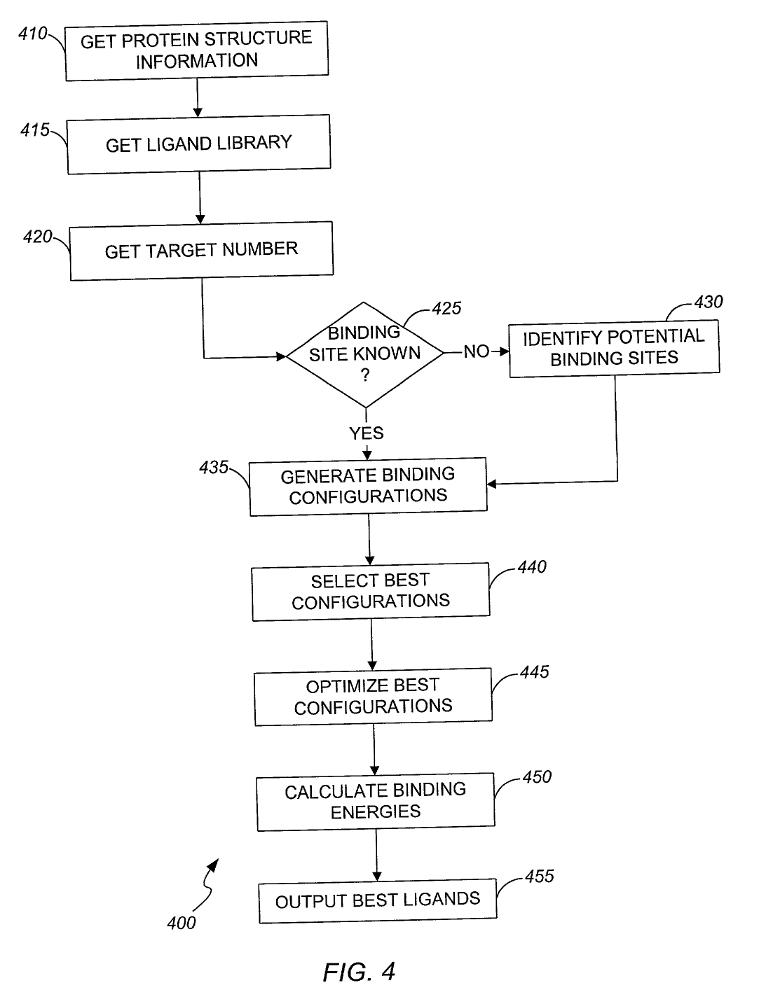

An alternate implementation providing a fast virtual screening protocol 400 for screening of large combinatorial libraries of drugs or other small molecules is illustrated in FIG. 4. Like the protocol of FIG. 2, method 400 begins by obtaining protein and ligand structural information (steps 410 and 415). In this implementation, the set of potential ligands is preferably large—for example, a combinatorial library of potential ligands derived from a publicly-available database of small molecules. In particular advantageous implementations, the ligand library can include 100, 1000, 10,000, or even 100,000 or more potential ligands, and structural information can be retrieved for each member of the ligand library in step 415. The method also obtains a target number (step 420), specifying a number of desired target ligands to be identified (e.g., for further experimental screening). The target number can be based on a number of factors, including, for example, the cost, throughput and effectiveness of experimental screening methods to be applied to the target ligands identified by method 400.

As described above in the context of FIG. 2, if the binding site for the protein is not known (the NO branch of step 425), method 400 uses the structural information to identify a probable binding site (e.g., according to steps 220-240 described above) (step 430). Initial conformations for each set of selected ligands in the known or predicted binding site are generated (step 435) using known techniques, such as the DOCK 4.0 package or any other publicly-available or in-house developed docking software. A set of best conformations is selected for each ligand in each potential binding area (step 440). For this implementation, where the set of potential ligands is large, the set of best conformations may include fewer conformations for each ligand than used in the implementations discussed above (e.g., approximately the best one percent or less than one percent of conformations for each ligand). Each of these conformations is energy minimized (step 445)—for example, using the conjugate gradient method. Binding energies are then calculated for each minimized conformation (step 450)—for example, using the energy scoring function of Equation (1), above, to account for salvation. The best ligands (i.e., those having the lowest predicted binding energy) are identified and output (step 455), for example, in the form of a data file identifying the specified target number of ligands having the lowest calculated binding energy for the protein.

In one application of this protocol the virtual screening of a library of fifty-four thousand seven hundred and eighty-three (54783) compounds was performed against a target protein of known 3D structure using the scoring function of Equation (1) to account for the salvation penalty for the ligand in calculating ligand binding affinities. This fast scanning protocol, which we have called Tera-Hier-Dock, was performed on 16 Silicon Graphics R10000 processors in 2 days. Five hundred best ligands were selected from the initial ligand library and tested experimentally for activity against the target. Eighteen (18) of those were confirmed as effective binders. This amounts to a 4% success rate in screening the starting library, a level of performance that could, for example, significantly shorten drug development timescales and costs, and increase efficiency in the development and optimization of lead compounds in drug-discovery processes.

The methods described above can be used to identify binding modes of a set of ligands for which affinity has been measured experimentally. The binding modes can be characterized using the amino acids that make direct electrostatic or vander Waal's contact with, or form hydrogen bonds with, the ligand and the importance of these residues in binding can be tested experimentally by point mutation studies. Once the critical amino acids that contribute to the binding of the ligand are identified, the distances between those amino acids and the bound ligand and between the amino acids themselves can be measured to generate a distance map. This distance map can be used according to known techniques to derive a pharmacophore model—a geometric model representing a pattern of features that are (or are predicted to be) required for binding with the protein.

The resulting pharmacophoric model can be used in conjunction with small molecule databases such as the Available Chemical Database (ACD) to search for compounds that fit the pharmacophore pattern, which may be promising lead compounds for drug development. Methods of deriving and using pharmacophores are described, for example, in Pharmacophore Perception, Development, and Use in Drug Design, Osman F. Güner, ed. (La Jolla, Calif. 2000: International University Line) and in U.S. Provisional Application No. 60/233,294, filed on Sep. 15, 2000, which is incorporated by reference herein. Pharmacophores generated according to the methods described herein can thus be used to screen large databases of small molecules and potentially to identify a large number of potential drug candidates for further virtual ligand screening according to others of the methods described herein.

The methods and apparatus described herein are broadly applicable for use in modeling docking interactions for any protein-ligand complex (including both naturally-occurring ligands and unnatural analogs) for which sufficient experimental or predicted structural information is known. The following examples illustrate the application of these techniques to the modeling of ligand binding interactions for several globular and transmembrane proteins. In one set of examples (Examples 1, 2 and 5), the protocol was performed to study globular proteins for which crystal structures with bound ligand are known. Using no information on the coordinates of the ligand bound to the crystal structure, the protocol was used to predict the ligand binding site (the geometry of which was compared to the known binding site configuration) and to calculate binding energies of the ligands in the binding site. In the second set of examples (Examples 3 and 4), a predicted structural model of two G-Protein coupled receptors was used to study the ligand binding properties of the corresponding proteins.

### EXAMPLE 1

### Docking Studies for Phenylalanyl t-RNA Synthetase

The protocol described above was tested on Phenylalanyl t-RNA synthetase (PheRS), as described below. Experimental results have identified phenylalanine analogs that are incorporated into the protein, as well as analogs that are not bound. Reshetnikova et al. have determined the crystal structure of the PheRS complexed with phenylalanine and PheRS complexed with phenylalaninyl-adenylate (PheOH-AMP) at 2.7 Å and 2.5 Å resolution, respectively. L. Reshetnikova, et al. (1999) J. Mol. Biol. 287, 555-568, which is incorporated by reference herein. Although the binding site of Phe in PheRS is known, the 3-D coordinates of phenylalanine bound to the phenylalanyl t-RNA synthetase were not used in this simulation. In this study we have used the crystal structure of phenylalanyl t-RNA synthetase with no ligand bound to it.

(1) Mapping possible binding regions: The negative image of the protein molecular surface was filled with a set of overlapping spheres. A probe of 1.4-Å radius was used to generate a 5 dots/A molecular surface. Sphere clusters were generated for the whole protein by using the program SPHGEN.

(2) Defining regions for docking: The sphere-filled volume representing the empty space inside the protein was divided into overlapping regions. In this case the binding region of phenylalanine to its t-RNA synthetase is known and hence this region was chosen for docking studies.

(3) Generating docked conformations of the receptor-ligand complexes: Orientations of the phenylalanine into the protein were generated by using DOCK 4.0 as described above, using flexible docking with torsion minimization of ligands, a nondistance-dependent dielectric constant of one, and a cutoff of 10 Å for energy evaluation. The conformations were ranked using energy scoring, and the top 10% of docking structures were carried in to step 4, below.

(4) Performing annealing MD for the complexes: Further optimization of ligand conformation in each binding region was performed using the annealing MD techniques described above. The annealing MD also leads to a better scoring function by using a full atom force field and salvation effects. The best 10 of the conformations generated in the preceding step were used in annealing MD simulations performed in 10 cycles of 1 ps at each temperature from 50 to 600 K, using the DREIDING force field, a nondistance-dependent dielectric constant of one, and a nonbond list cutoff of 9 Å. The best conformers of the complexes from annealing were submitted to energy minimization.

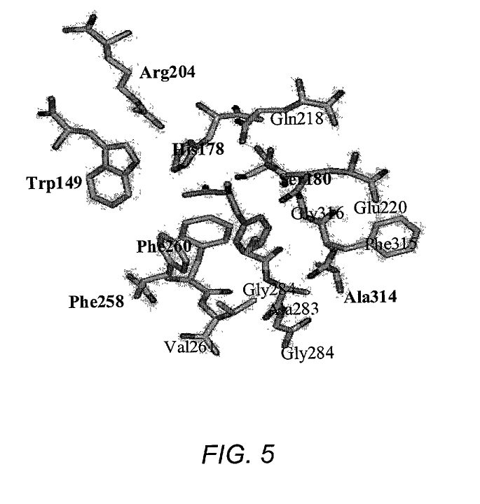

(5) Selecting the best binding site conformation: The minimized conformations were scored using the DREIDING forcefield and Surface Generalized Born salvation model and equation (2). The best conformation that leads to the lowest binding energy was selected as the predicted binding site of phenylalanine to phenylalanyl t-RNA synthetase. The protocol predicts the bound structure of phenylalanine in PheRS with a precision of 0.62 Å in RMS of the coordinates of all the atoms in the ligand (phenylalanine) from the known crystal structure. The predicted structure for the probable binding site is shown in FIG. 5. The predicted structure is 0.62 Å in CRMS deviation from the known crystal structure.

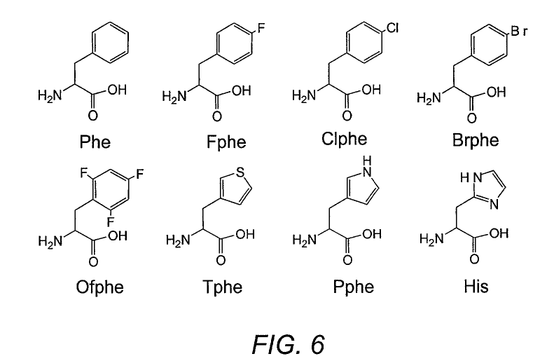

(6) Docking all other ligands into the binding site: Phenylalanine analogs 4-fluoro-phenylalanine (Fphe), 4-chlorophenylalanine (Clphe), 4-bromo-phenylalanine (Brphe), 2,4,6-trifluoro-phenylalanine (Ofphe), 3-thienylalanine (Tphe), 3-pyrrolylalanine (Pphe) and histidine (His) (shown in FIG. 6) were docked into the binding site by repeating steps 3-5 for each ligand. Binding energies were calculated using equation 2, above. The binding energies were computed using the DREIDING forcefield and cell multipole method for non-bonds, according to H. Q. Ding, et al. (1992) J. Chem. Phys. 97, 4309, which is incorporated by reference herein. The charges for the ligands were assigned using the charge equilibration method.

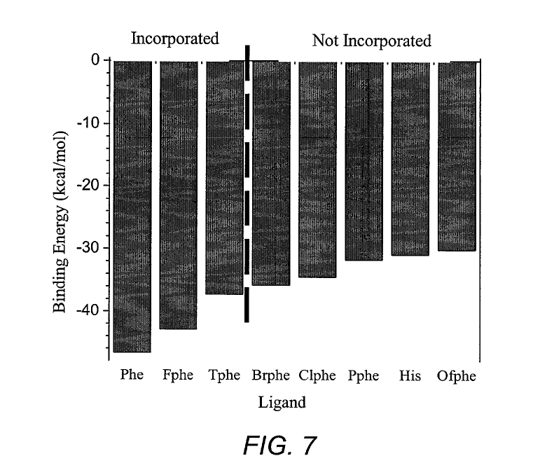

(7) Ranking ligand affinities by using binding energies. The binding energies for the best complexes were calculated using equation (2). The binding energies corresponding to different ligands were then compared and ordered. The ligands that have more favorable binding energies having higher affinities to the protein. The results for the series of ligands are shown in FIG. 7, which shows the binding energies, in kcal/mol, calculated for the various analogs of FIG. 6 in the binding pocket of PheRS illustrated in FIG. 5. Substrates to the left of the vertical line can be incorporated in protein in wild-type E. coli cells; those to the right cannot. Predicted binding energies for those analogs that are taken up in vivo are lower than that of those analogs that are not taken up in the protein synthesis. The predicted difference in binding energy between Fphe and Phe is 3.79 kcal/mol. Measurements from in vitro experiments give a value of 6 to 8 kcal/mol.

### EXAMPLE 2

### Docking Studies for Histdyl t-RNA Synthetase

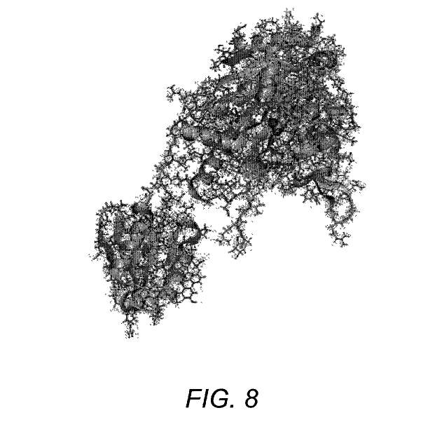

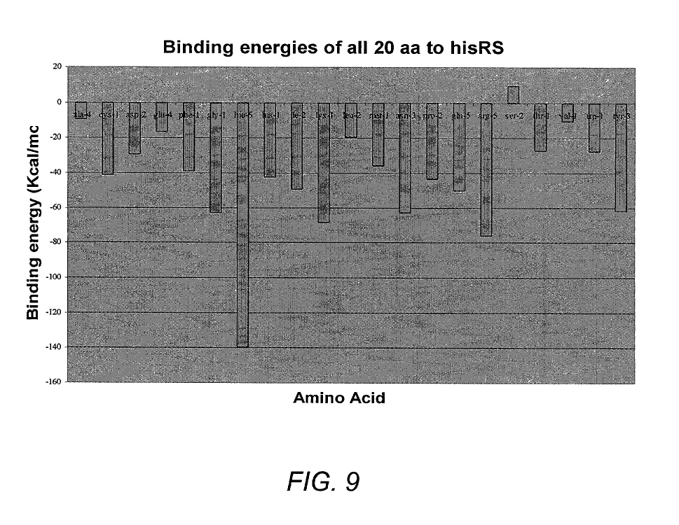

The protocol of Example 1 was repeated to simulate the binding of twenty-one amino acid ligands, including the protonated and unprotonated forms of histidine as well as the other nineteen naturally-occurring amino acids, in histdyl t-RNA synthetase (starting from the protein's known crystal structure, but without using the known binding site in the simulations). The predicted binding site for the enzyme is illustrated in FIG. 8, and shows a 0.39 Å in CRMS deviation from the crystal structure. The calculated binding energies are illustrated in FIG. 9. The protonated form of histidine shows the best binding energy, consistent with experimental observations that histidine is the only ligand recognized by this enzyme.

### EXAMPLE 3

### Docking Studies for OR S25

The binding of odorants like aliphatic alcohols and acids to olfactory receptor S25 (OR S25) was studied without any previous knowledge of the binding site. Olfactory receptors interact with molecules of widely different structures and are therefore expected to exhibit high structural diversity in the ligand-binding region. Hyper-variable regions in ORs have been identified in transmembrane domains (TMs) 3-5, as reported by Y. Pilpel, et al. (1999) Protein Sci. 8, 969-977; M. S. Singer, et al. (1995) Recept. Channels 4, 141-147; P. Mombaerts (1999) Science 286, 707-711; and L. Buck, et al. (1991) Cell 65, 175187, and are thought to be involved in odorant binding. For OR S25, studies have pointed to an odor-binding pocket composed of residues from TMs 3-7. D. Krautwurst, et al. (2000), Cell 95, 917-926 (1998); M. S. Singer, Chem. Senses 25, 155-165; R. P. Poincelot, et al. (1970) Biochemistry 9, 1809-1816; M. S. Singer, et al. (1994) NeuroReport 5, 1297-1300, each of which is incorporated by reference herein. Nevertheless, the exact location of the binding site is not known.

A complete scanning of all possible docking regions for S25 was performed for 24 potential ligands according to the protocol set out above, as follows, starting from a predicted structure of OR S25 calculated as described in the co-pending U.S. application Ser. No. ______, titled “Methods and Apparatus for Predicting Structure of G-Protein Coupled Receptors,” to N. Vaidehi, et al., filed on Mar. 22, 2001, incorporated by reference above.

(1) Mapping possible binding regions. The negative image of the receptor molecular surface was filled with a set of overlapping spheres. A probe of 1.4-Å radius was used to generate a 5 dots/Å molecular surface. Sphere clusters were generated for the whole receptor by using the program SPHGEN.

(2) Defining regions for docking. The sphere-filled volume representing the empty space inside the receptor was divided into five overlapping regions, covering the extracellular portion of the receptor, as well as ⅔ of the inside of the helical barrel. Regions expected to be in contact with the membrane or involved in binding with the G protein were excluded from docking.

(3) Generating docked conformations of the receptor-ligand complexes. The study included 24 aliphatic alcohols, carboxylic acids, dicarboxylic acids, and bromocarboxylic acids containing 4-9 carbon atoms for which data on odor response preferences for several mouse olfactory receptors has been reported by Malnic et al. Among the odorants in that list, S25 responds positively to hexanol and heptanol only. Accordingly, a complete scanning of all possible docking regions for S25 was first performed with the alcohol series.

Each ligand was built in the extended conformation. The starting conformations were optimized by minimization of the potential energy by using the conjugate gradient method with DREIDING force field and Gasteiger charges as described above. The minimized conformations were used as starting conformations for docking. The acids were considered in their protonated forms for docking because the pH range in the human nasal mucus is between 6 and 7 in normal individuals, as reported by A. Sachdeva, et al. (1993) Indian J. Med. Res. B 98, 265-268. The salvation energies for the ligands were calculated by using Poisson-Boltzmann continuum solvent model with the JAGUAR program. The solvation energies of the acids were calculated for the deprotonated species, because they are the dominant form in solution.

Orientations of the ligands into the receptor were generated by using DOCK 4.0 as described above, using flexible docking with torsion minimization of ligands, a nondistancedependent dielectric constant of one, and a cutoff of 10 Å for energy evaluation. The conformations were ranked using energy scoring. The best 10-30 conformations for each ligand in each possible binding region were used as input for the annealing molecular dynamics step. 110-120 conformations were generated for each ligand in a total of 700 conformations covering the receptor space available for docking.

(4) Performing annealing MD for the complexes. Further optimization of ligand conformation in each binding region was performed using the annealing MD techniques described above. The annealing MD also leads to a better scoring function by using a full atom force field and salvation effects. All conformations generated in the preceding step were used in annealing MD simulations performed in 10 cycles of 1 s from 50 to 600 K, using the DREIDING force field, a nondistance-dependent dielectric constant of one, and a nonbond list cutoff of 9 Å. The best conformers from annealing were submitted to energy minimization.

(5) Selecting the best conformation and probable binding site. The conformations that have the lowest energy scores (determined using Equation 2, above) were selected. These exhibit a preferential region for binding.

(6) Redocking into the binding site. To obtain a comparative score for all ligands in the most possible binding site, a 10×5×5 Å box was identified enclosing the best conformations for butanol to heptanol. Steps 3-5 were then repeated for the alcohol and acid series.

(7) Cross-evaluating conformation energies by using perturbation techniques. The lowest energy conformations among the alcohols were used as template to build other members of the alcohol series. These complexes then were submitted to annealing MD to ensure that every ligand was evaluated in the same orientation starting from the best conformation of others.

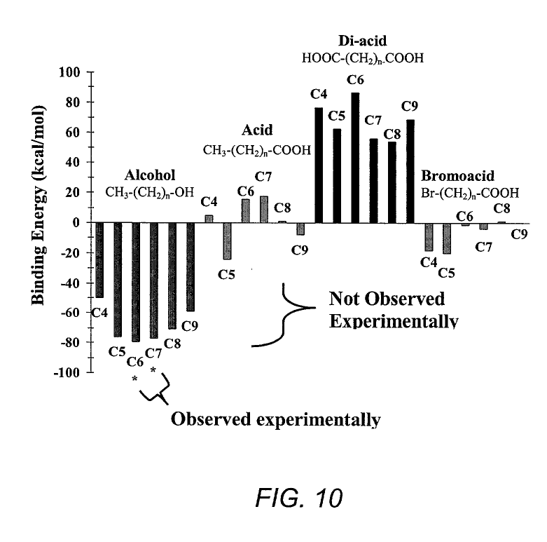

(8) Ranking ligand affinities by using binding energies. The binding energies for the best complexes were calculated as the difference in the ligand energy in the receptor and in solution (using Equation 2, above). The binding energies corresponding to different ligands were then compared and ordered, the ligands for which the receptor-ligand complex has more favorable binding energies having higher affinities to the receptor. The results for the series of ligands are shown in FIG. 10 (with binding energy bars shaded according to the chemical class listed above each bar; in each case, the number following the letter “C” indicates the number of carbon atoms).

These results correlate well with the experimental observations of Malnic et al. Thus, hexanol and heptanol, the two compounds predicted by the OR S25 structure to have the highest binding energies, were the only two compounds that elicited measurable responses in the experiments. Significantly, the model predicts affinities for other, less avidly bound compounds that may activate the receptor but are below the experimental detection threshold. For example, the structural model predicts that pentanol would have the third best binding energy, only 1.3 kcal/mol less favorable than heptanol. Because the responses observed for hexanol and heptanol were near threshold, binding studies of such ligands at higher concentrations or in other assay systems might show a response and would test the predicted energetics.

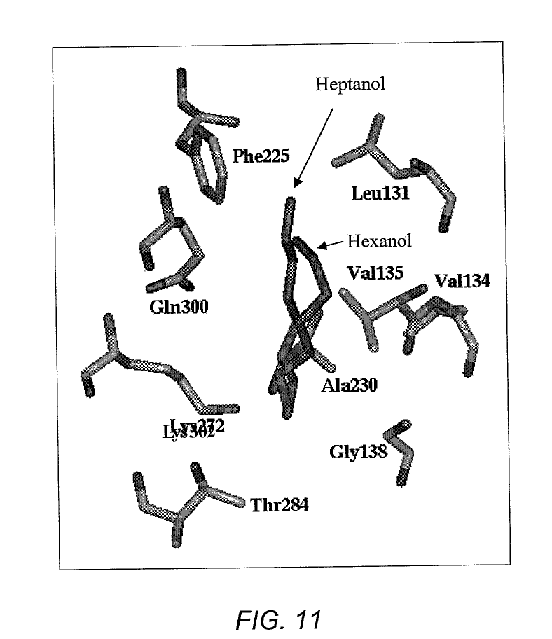

The predicted binding pocket for the preferred ligands hexanol and heptanol is shown in FIG. 11. The pocket is situated between TMs 3-7, approximately 10 A deep from the extracellular surface. These results suggest that TMs 3, 5, and 6 have residues directly involved in binding. TM4 may have an important role in binding as it packs against TM3 and TM5 and therefore can alter their relative position if key residues of TM4 are mutated. Fifteen residues are predicted to constitute the hexanol-heptanol binding site. These residues are variable in the sequence alignment of ORs according to Malnic et al., consistent with their involvement in differential odor binding for different OR subtypes.

Lys-302, which hydrogen bonds to the hydroxyl moiety, appears to be critical for alcohol binding by OR S25. Substitutions in this residue could switch receptor specificity toward other functional groups. Hydrophobic residues Phe-225, Leu-131, Val-134, Val-135, and Ala-230 formed Van der Waals contacts with the ligand, accounting for the specificity of the OR S25 model for 6-7 carbon compounds. Hydrophobic substitutions in these residues would be expected to modulate the preferred carbon length. In particular, the model predicts that substitutions of Val for Phe-225 and Val for Leu-131 would be expected to create more space in the pocket and shift its specificity toward larger ligands. Substitutions of Phe for Leu-131 would be predicted to have the opposite effect. Polar residues Thr-284 and Gln-300 were also in close contact with the ligand but did not appear to contribute any hydrogen bonding specificity. These residues could be important for interactions of other compounds with OR S25.

### EXAMPLE 4

### Docking Studies for OR S18

The protocol of Example 3 was repeated, with some variations, to simulate the binding of the same series of alcohols and acids with a different olfactory receptor, S18. The variations used in this Example were as follows:

(1) DOCK 4.0 was used in database mode to generate of conformations for each ligand in each binding region;

(2) Best ligand conformations for each binding region were selected based on the percentage of buried surface of the ligand as a first criterion, with energy scoring as a second criterion performed only on conformations in which at least 70% of the ligand surface area was calculated to be buried within the protein;

(3) Annealing molecular dynamics was omitted during the initial search for the probable binding site (i.e., step 4 as set out in Example 3). However annealing molecular dynamics was used in the redocking steps (in particular step 7 as set out in Example 3);

(4) The salvation method used in calculated the binding energies was FSM as implemented in the program MPSim described above.

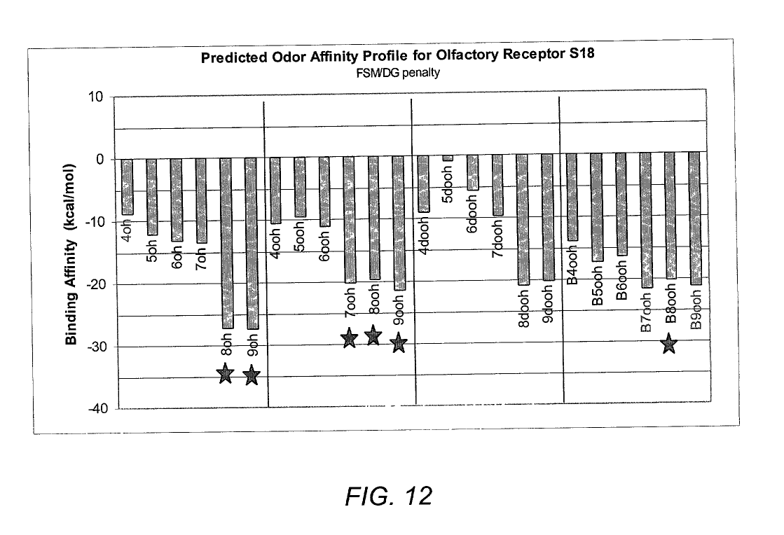

The binding affinity profile for olfactory receptor S18 is illustrated in FIG. 12. This receptor responds experimentally to octanol, nonanol, heptanoic acid, octanoic acid, nonanoic acid and 8-bromo-octanoic acid according to Malnic et al. The predicted odor affinity profile agrees with the experimental responses within each chemical class. It also predicts that the untested 7-bromo-heptanoic acid and 9-bromononanoic acid should also elicit response from S18.

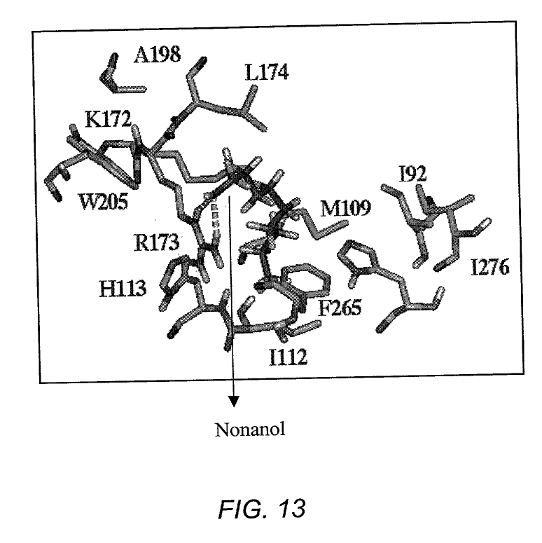

The predicted binding site for nonanol in S18 is shown in FIG. 13. Nonanol is anchored to the binding site through a hydrogen bond to Arg173. Residues predicted as involved in binding are (TM stands for transmembrane domain and EC is extracellular loop): Ile92 (EC1), Met109 (TM3), Ile112 (TM3), His113 (TM3), Lys172 (TM4), Arg173 (EC2), Leu 174 (EC2), Ala198 (EC2), Trp205 (TM5), Phe265 (TM6), Ile276 (EC3), and His279 (EC3). Mapping the residues predicted as involved in binding into the sequence alignment of olfactory receptors S25 and S18 suggests that transduction of odorant binding into electrical signal to the brain involves relative movement of TMs 3 and 6. Although this suggestion needs further investigation, it illustrates the kinds of information that can be derived from computer-generated models of protein-ligand complexes of the type described herein.

### EXAMPLE 5

### Prediction of the Binding Site for Retinal in Bovine Rhodopsin

The protocol in FIG. 2 was used to predict the binding of retinal to bovine rhodopsin, using the three-dimensional structure experimentally determined by x-ray diffraction at 2.80 □ resolution, as described in K. Palczewski et al. (2000) Science 289, 739. Although the location of the binding site is known from the crystal structure, that information was not used. Instead, the empty volume available for docking in the receptor was scanned for possible binding sites as described for olfactory receptor S18. A combined percentage of buried surface area and energy criteria was used to select the best conformations of trans-retinal and cis-retinal, each independently docked into six (6) overlapping docking regions. The selected conformations were used to define the binding site for redocking as described above.

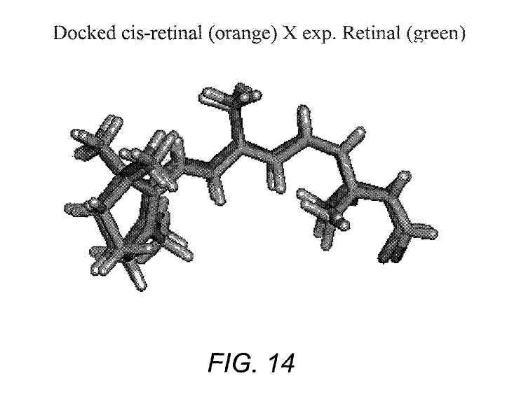

The location of the predicted binding site for retinal coincides with the experimental location within 3.4 □ rms deviation. The superposition of the rhodopsin-retinal complexes as determined in the crystal structure and as predicted by the above described protocol is shown for cis-retinal in FIG. 14.

The invention can be implemented in digital electronic circuitry, or in computer hardware, firmware, software, or in combinations of them. Apparatus of the invention can be implemented in a computer program product tangibly embodied in a machine-readable storage device for execution by a programmable processor; and method steps of the invention can be performed by a programmable processor executing a program of instructions to perform functions of the invention by operating on input data and generating output.

The invention can be implemented advantageously in one or more computer programs that are executable on a programmable system including at least one programmable processor coupled to receive data and instructions from, and to transmit data and instructions to, a data storage system, at least one input device, and at least one output device. Each computer program can be implemented in a high-level procedural or object-oriented programming language, or in assembly or machine language if desired; and in any case, the language can be a compiled or interpreted language. If the various techniques are implemented in multiple computer programs, the protocols can be implemented as a script, such as a PERL script that executes different parts of the protocol in a serial fashion.

Generally, a processor will receive instructions and data from a read-only memory and/or a random access memory. Generally, a computer will include one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM disks. Any of the foregoing can be supplemented by, or incorporated in, ASICs (application-specific integrated circuits).

A number of implementations of the invention have been described. Nevertheless, it will be understood that various modifications may be made without departing from the spirit and scope of the invention. Accordingly, other embodiments are within the scope of the following claims.

What is claimed is:

1. A computer implemented method for modeling ligand-protein binding interactions, comprising:
providing structural information describing the structure of a protein and a set of one or more ligands;
using the structural information for the protein to identify a binding region of the protein;
identifying a plurality of preferred binding conformations for each of the set of ligands in the binding region;
optimizing the preferred binding conformations using annealing molecular dynamics, the annealing molecular dynamics including salvation effects;
calculating a binding energy for each of the set of ligands in the corresponding optimized preferred binding conformations; and
selecting for each of the set of ligands the lowest calculated binding energy in the optimized preferred binding conformations, and outputting the selected calculated binding energies as the predicted binding energies for each of the set of ligands.

2. The method of claim 1, wherein:
the binding region is a known binding region defined by the structural information.

3. The method of claim 1, wherein:
the binding region is an unknown binding region; and
using the structural information for the protein to identify a binding region of the ligand in the protein comprises predicting a probable binding region based at least in part on the structural information.

4. The method of claim 3, wherein predicting a probable binding region comprises:
mapping the empty volumes available for ligand binding in the protein to identify one or more potential binding regions;
generating initial conformations for one or more ligands known to bind the protein using docking techniques in each of the one or more potential binding regions;
selecting from the initial conformations for each of the known ligands a plurality of best conformations in each of the potential binding regions and scoring an energy function for each of the best conformations; and
identifying the probable binding site based on a spatial location of the conformations having the lowest energy scores.

5. The method of claim 4, further comprising:
before scoring the energy function for each of the best conformations, optimizing the selected best conformations to obtain a set of energy-minimized conformations for each of the known ligands in each of the potential binding regions;
wherein the energy function is scored for each of the energy-minimized conformations.

6. The method of claim 4, further comprising:
before scoring the energy function for each of the best conformations, calculating for each of the best conformations a percentage of the ligand surface area buried within the protein for the conformation;
wherein the energy function is scored only for a subset of the best conformations having a calculated percentage of the ligand surface area buried within the protein exceeding a predetermined surface area threshold.

7. The method of claim 1, wherein identifying the preferred binding conformations for each of the set of ligands comprises:
generating initial conformations for each of the set of ligands in the binding region using docking techniques; and
selecting from the initial conformations for each of the ligands a plurality of best conformations.

8. The method of claim 7, further comprising:
after selecting the best conformations, optimizing the selected best conformations to obtain a set of energy-minimized conformations for each of the ligands;
wherein the preferred binding conformations comprise the energy-minimized conformations.

9. The method of claim 1, wherein:
the annealing molecular dynamics includes a full atom force field.

10. The method of claim 1, wherein:
the solvation effects include a continuum description of solvation.

11. The method of claim 1, wherein:
the salvation effects include a surface-area based salvation model.

12. The method of claim 1, wherein:
calculating a binding energy for each of the set of ligands includes taking the difference in the ligand energy in the receptor and in solution.

13. The method of claim 1, wherein:
the binding energy is calculated for a ligand according to a scoring function comprising subtracting the free energy of the ligand in water from the energy of the ligand in the protein.

14. The method of claim 1, wherein:
the binding energy is calculated for a ligand according to a scoring function comprising subtracting the free energy of the protein and the free energy of the ligand from the free energy of the ligand in the protein.

15. The method of claim 1, further comprising:
identifying from the set of ligands one or more ligands predicted to have high binding affinity based on the calculated binding energy of the ligands in the binding site.

16. The method of claim 1, wherein:
the protein is a globular protein or a transmembrane protein.

17. A computer-implemented method for predicting the structure of a protein binding site for a protein having an unknown binding site, the method comprising:
providing structural information describing the structure of a protein having an unknown binding site and a set of one or more ligands known to bind to the protein;
using the structural information for the protein to identify a plurality of potential binding regions of the protein;
generating initial conformations for one or more of the ligands using docking techniques in each of the potential binding regions;
selecting from the initial conformations for each of the ligands a plurality of best conformations in each of the potential binding regions and scoring an energy function for each of the best conformations;
identifying the probable binding site based on a spatial location of the conformations having the lowest energy scores; and
outputting structure information describing the three-dimensional structure of the probable binding site.

18. The method of claim 17, further comprising:
before scoring the energy function for each of the best conformations, optimizing the selected best conformations to obtain a set of energy-minimized conformations for each of the ligands in each of the potential binding regions;
wherein the energy function is scored for each of the energy-minimized conformations.

19. The method of claim 17, further comprising:
before scoring the energy function for each of the best conformations, calculating for each of the best conformations a percentage of the ligand surface area buried within the protein for the conformation;
wherein the energy function is scored only for a subset of the best conformations having a calculated percentage of the ligand surface area buried within the protein exceeding a predetermined surface area threshold.

20. A computer-implemented virtual screening method for screening a ligand library, the method comprising:
receiving protein structural information describing the structure of a protein;
receiving ligand structural information describing the structure of a plurality of ligands in a ligand library;
receiving an input specifying a desired number of candidate ligands to be identified in the ligand library;
using the structural information for the protein to identify a binding region of the protein;
generating a set of initial binding conformations for each of the ligands in the binding region;
calculating an energy function for each of the initial binding conformations and selecting for each of the ligands a plurality of the initial binding conformations having the lowest calculated energy as a set of best conformations;
optimizing the best conformations;
calculating a binding energy for each of the ligands in the corresponding optimized best conformations; and
selecting from the plurality of ligands a set of the desired number of candidate ligands having the lowest calculated binding energy in the optimized best binding conformations, and outputting the selected set of candidate ligands.

21. The method of claim 20, wherein:
the plurality of ligands comprises at least 500 ligands.

22. The method of claim 20, wherein:
the plurality of ligands comprises at least 1,000 ligands.

23. The method of claim 20, wherein:
the plurality of ligands comprises at least 5,000 ligands.

24. The method of claim 20, wherein:
the plurality of ligands comprises at least 10,000 ligands.

25. The method of claim 20, wherein:
the plurality of ligands comprises at least 50,000 ligands.

26. The method of claim 20, wherein:
the plurality of ligands comprises at least 100,000 ligands.

27. The method of claim 20, wherein:
calculating a binding energy for each of the set of ligands includes taking the difference in the ligand energy in the receptor and in solution.

28. The method of claim 20, wherein:
the binding energy is calculated for a ligand according to a scoring function comprising subtracting the free energy of the ligand in water from the energy of the ligand in the protein.

29. A computational model of a ligand-protein complex for a protein having an unknown binding site, the model comprising:
a computer-readable memory storing data describing an optimized preferred binding conformation for the protein and a ligand known to bind to the protein, the optimized binding conformation being generated according to the method claim 1.

30. A computational model of a predicted structure for a protein binding site for a protein having an unknown binding site, the model comprising:
a computer-readable memory storing data describing the three-dimensional structure of the probable binding site for the protein generated according to the method claim 15.

31. A computer program product on a computer-readable medium for modeling ligand-protein binding interactions, the computer program product comprising instructions operable to cause a programmable processor to:
provide structural information describing the structure of a protein and a set of one or more ligands;
use the structural information for the protein to identify a binding region of the protein;
identify a plurality of preferred binding conformations for each of the set of ligands in the binding region;
optimize the preferred binding conformations using annealing molecular dynamics, the annealing molecular dynamics including salvation effects;
calculate a binding energy for each of the set of ligands in the corresponding optimized preferred binding conformations; and
select for each of the set of ligands the lowest calculated binding energy in the optimized preferred binding conformations, and output the selected calculated binding energies as the predicted binding energies for each of the set of ligands.

32. A computer program product on a computer-readable medium for predicting the structure of a protein binding site for a protein having an unknown binding site, the computer program product comprising instructions operable to cause a programmable processor to:
provide structural information describing the structure of a protein having an unknown binding site and a set of one or more ligands known to bind to the protein;
use the structural information for the protein to identify a plurality of potential binding regions of the protein;
generate initial conformations for one or more of the ligands using docking techniques in each of the potential binding regions;
select from the initial conformations for each of the ligands a plurality of best conformations in each of the potential binding regions and score an energy function for each of the best conformations;
identify the probable binding site based on at least one of a percentage surface area of the ligand buried in the protein or a spatial location of the conformation having the lowest energy score; and
output structure information describing the three-dimensional structure of the probable binding site.

33. A computer program product on a computer-readable medium for screening a ligand library, the computer program product comprising instructions operable to cause a programmable processor to:
receive protein structural information describing the structure of a protein;
receive ligand structural information describing the structure of a plurality of ligands in a ligand library;
receive an input specifying a desired number of candidate ligands to be identified in the ligand library;
use the structural information for the protein to identify a binding region of the protein;
generate a set of initial binding conformations for each of the ligands in the binding region;
calculate an energy function for each of the initial binding conformations and selecting for each of the ligands a plurality of the initial binding conformations having the lowest calculated energy as a set of best conformations;
optimize the best conformations;
calculate a binding energy that includes salvation for each of the ligands in the corresponding optimized best conformations; and
select from the plurality of ligands a set of the desired number of candidate ligands having the lowest calculated binding energy in the optimized best binding conformations, and output the selected set of candidate ligands.

34. A computer-implemented method of generating a pharmacophore, comprising:
providing structural information describing the structure of a protein and a set of one or more ligands known to bind to the protein;
using the structural information for the protein to identify a binding region of the protein;
identifying a plurality of preferred binding conformations for each of the set of ligands in the binding region;
optimizing the preferred binding conformations using annealing molecular dynamics, the annealing molecular dynamics including salvation effects;
calculating a binding energy for each of the set of ligands in the corresponding optimized preferred binding conformations;
selecting for each of the set of ligands the optimized preferred binding conformation having the lowest calculated binding energy;
generating a pharmacophore model based at least in part on the selected optimized preferred binding conformations, the pharmacophore model defining a pattern of ligand features predicted to be required for binding to the protein; and
outputting data representing the pharmacophore model for use in drug design.

35. The method of claim 34, further comprising:
using the pharmacophore model as a template to search a chemical information database to identify one or more molecules predicted to bind to the protein.
